(19) United States
(12) Patent Application Publication
(10) Pub. No.: US 2013/0004604 A1
(43) Pub. Date: Jan. 3, 2013

(54) DARK CHOCOLATE DELIVERY SYSTEM FOR A COMBINATION OF DIETARY SUPPLEMENTS AND PHARMACEUTICALS

(75) Inventor: Judy Davis, San Clemente, CA (US)

(73) Assignee: HERO NUTRITIONAL PRODUCTS LLC, San Clemente, CA (US)

(21) Appl. No.: 13/491,456

(22) Filed: Jun. 7, 2012

Related U.S. Application Data
(63) Continuation of application No. PCT/US10/59890, filed on Dec. 10, 2010.
(60) Provisional application No. 61/285,280, filed on Dec. 10, 2009.

(51) Int. Cl. A61K 47/46; A23G 1/32; A23L 1/302; A61P 3/14; A61K 36/28; A61P 25/00; A61P 3/02; A23G 1/42; A23L 1/304

(52) U.S. Cl. 424/764; 426/61; 426/71; 426/72; 426/74; 426/631; 426/519; 426/414; 426/520; 426/311; 514/777

(57) ABSTRACT

An edible, chewable dark chocolate candy for the oral delivery of dietary supplements and pharmaceutical compounds. The-chocolate formed or molded solid candy contains at least one active ingredient that is blended with the chocolate candy during conching of the chocolate candy. A method for manufacturing the chewable chocolate candy is also disclosed. In the blending phase, a chocolate liquor is blended with additional ingredients to obtain the desired individual chocolate recipe for the chocolate delivery vehicle. The selected dietary supplement and/or pharmaceutical compounds are then added to the chocolate liquor, which is then conched for the desired time period. When the conching phase is completed, the conched chocolate is tempered, after which the tempered chocolate having 60% or more cacao mass may be formed into various sizes and shapes for individually-wrapped pieces, or formed into large blocks for storage and shipment.

### RELATED APPLICATIONS

This application is a continuation of PCT International Patent Application No. PCT/US2010/059890, filed on Dec. 10, 2010, which claims priority from U.S. Provisional Application No. 61/285,280, filed on Dec. 10, 2009, entitled “Dark Chocolate Delivery System for a Combination of Dietary Supplements.” Each of these applications is incorporated herein by reference in its entirety.

### BACKGROUND OF THE INVENTION

1. Field of the Invention

The invention relates generally to a chewable chocolate delivery system, and more particularly, to a chewable dark chocolate candy for the oral delivery of a combination of dietary supplements and pharmaceutical compounds, and a method for manufacturing the same.

2. Related Art

The demand for dietary supplements for adults and children is constantly increasing and as this demand has grown, many new products are being introduced to the consumer market. In addition to the traditional multivitamins and minerals, such as iron, calcium, selenium, etc., various botanicals, herbs, and nutraceuticals are also being packaged and sold. All of these various dietary supplements may be sold in various combinations, some of which may be designed to provide a specific desired effect to the consumer. For example, a combination of dietary supplements may be designed to soothe and calm the consumer and provide stress relief, while another may assist the consumer in maintaining a healthy lifestyle and a desired weight.

In similar demand, over-the-counter (OTC) and prescription drugs play a vital role in America's health care system. While both are regulated by the U.S. Food and Drug Administration (FDA), OTC drug products are available to consumers without a prescription. There are more than 80 therapeutic categories of OTC drugs, ranging from acne drug products to weight control products, and over 100,000 OTC drug products are currently being sold in this country. Thus, OTCs are critical to our country's health care system because they provide easy access to certain drugs that can be used safely without the help of a health care practitioner.

Despite their importance, ingestion of OTC and prescription drugs can be, for many, an unpleasant experience. Most individuals take these drugs orally and may take several each day. Drugs that are taken orally generally come in the form of a liquid, a tablet, a pill, or a capsule. In many cases, this is not an enjoyable experience because the pharmaceuticals are either difficult to ingest due to size or texture, or are not palatable.

Previously, a selection of vitamin supplements has been made available for children in a chewable candy form that is pleasant tasting and readily palatable. Another delivery system having great potential is chocolate in its many forms. Chocolate has become one of the most popular flavors with consumers around the world, in the form of candy bars and smaller individually-wrapped pieces, and chocolate may potentially have its own beneficial health effects.

In general, chocolate is created from the cocoa bean, which is harvested, fermented, and dried, and then shipped to a chocolate manufacturing facility. At the chocolate manufacturers, the beans are cleaned, roasted, and graded. The shell is then removed, resulting in cacao nibs, which are then ground into a thick creamy paste, known as chocolate liquor or cocoa paste. The next step in the process is blending, where the chocolate liquor may be mixed with more cocoa butter, sugar, milk or milk powder, and vanilla. It is in this step where the various types of chocolate are made: dark chocolate, milk chocolate (by adding milk or milk powder), and white chocolate (having no chocolate liquor, only cocoa butter). The blended chocolate is then conched, tempered, molded, and wrapped for eventual distribution to consumers.

In particular, dark chocolate, sometimes called “plain chocolate” or “black chocolate”, generally refers to chocolate that is produced without the addition of milk or milk powder. Dark chocolate has a more pronounced chocolate taste than milk chocolate because it does not contain milk solids to compete with the chocolate taste. However, the lack of milk additives also means that dark chocolate is more prone to a dry, chalky texture and a bitter aftertaste. The basic ingredients in dark chocolate bars are cacao beans, sugar, an emulsifier such as soy lecithin to preserve texture, and a sweetener or flavoring agent such as vanilla. Dark chocolate is often distinguished by the percentage of cocoa solids in the bar. The cocoa content of commercial dark chocolate bars in the United States can range from 35% (sweet dark) to 70%, 75%, or even above 80% for extremely dark bars; however, the standard range of cocoa content for dark chocolates varies from country-to-country. Common terms used to distinguish the cocoa content of dark chocolate bars include bittersweet, semi-sweet, and sweet dark chocolate.

As noted earlier, some studies purportedly show chocolate to have its own beneficial health effects. These health benefits may be from antioxidants in the chocolate that help keep the cardiovascular system running well, as well as lower blood pressure and cholesterol. Chocolate also contains ingredients that act as an anti-depressant and a stimulant. These healthy properties are derived from the cacao bean and thus there are many more antioxidants in dark chocolate than milk or white chocolate.

For example, dark chocolates with high cocoa content contain more flavonoids—polyphenol compounds also found in apples, green tea and red wine that have various beneficial biochemical and antioxidant effects. Recent studies have shown that flavonoids may have antiviral, anti-allergic, antiplatelet, anti-inflammatory, antitumor, and antioxidant effects, and chocolate's flavonoids, in particular, may lower cholesterol build-up in the arteries. Thus, the proponents of chocolate's benefits generally believe that chocolates with higher cocoa content yield more health benefits and recommend dark chocolate of at least 75% cacao content, although some recommend 85% or higher. Dark chocolate with 60% cacao is known as bittersweet or extra dark chocolate and is commonly found in readily available commercial candy bars.

To date, no one has utilized dark chocolate candy as a delivery system for combinations of dietary supplements and pharmaceuticals, and because of chocolate's popularity world-wide, there is a need for a dark chocolate delivery system for various combinations of botanicals, minerals, vitamins, nutraceuticals, soluble and insoluble fibers, pharmaceuticals, probiotics, prebiotics, amino acids, digestive enzymes, and other nutritional supplements.

### SUMMARY OF THE INVENTION

A delivery system is provided for the oral delivery of a combination of dietary supplements, pharmaceuticals, and other nutritional ingredients. The delivery system includes dark chocolate in the form of a molded or formed solid candy, which may be in the form of a bar or bite-size piece.

According to one implementation, the edible, chewable composition includes a delivery system for delivering a blend of dietary supplements and/or pharmaceutical compounds to a user's body. The drug delivery system includes a delivery vehicle in the form of a molded or formed solid dark chocolate candy, and a pharmaceutical and/or nutritional supplement as an active ingredient contained within the chocolate candy. In particular, the delivery vehicle may include dark chocolate having at least 60% cacao mass that has been blended, conched, and tempered. The chocolate may also include natural and/or artificial flavors, colors, and preservatives.

The active ingredient may include a blend of dietary supplements to provide a desired effect on the user. Such dietary supplements may include nutraceuticals (i.e., an extract of food purported to have a medicinal effect on human health) such as botanical and herbal extracts and antioxidants, or any combination of food supplements such as vitamins, minerals, soluble and insoluble fibers, herbs, plants, amino acids, probiotics, prebiotics, digestive enzymes, or any other health promoting ingredient. In other implementations, the active ingredient may include a pharmaceutical, such as OTC or a prescription drug, as the primary ingredient or in combination with a dietary supplement.

According to another implementation, a method of preparing a chewable solid dark chocolate candy containing an active ingredient is provided. The method includes preparing a chocolate liquor by blending the necessary ingredients for the desired type of chocolate, adding the active ingredient to the chocolate liquor, conching the chocolate liquor with the active ingredient to achieve the desired taste and texture, and then tempering the chocolate. After tempering, the chocolate may be stored in large blocks for later processing, or molded or formed into smaller pieces and individually wrapped.

According to yet another implementation, a method of preparing a chewable solid dark chocolate candy containing an active ingredient is provided. This method includes reheating a block of tempered dark chocolate, adding at least one active ingredient to the reheated chocolate, blending the reheated chocolate with the active ingredient to achieve the desired taste and texture, and then tempering the blended chocolate. After tempering, the chocolate may be stored in large blocks for later processing, or molded or formed into smaller pieces and individually wrapped.

Other compositions, systems, methods, features and advantages of the invention will be or will become apparent to one with skill in the art upon examination of the following figure(s) and detailed description. It is intended that all such additional compositions, systems, methods, features and advantages be included within this description, be within the scope of the invention, and be protected by the accompanying claims.

### BRIEF DESCRIPTION OF THE FIGURES

The invention may be better understood by referring to the following figure(s). The components in the figure(s) are not necessarily to scale, emphasis instead being placed upon illustrating the principles of the invention. In the figure(s), like reference numerals designate corresponding parts throughout the different views.

FIG. 1 shows a flow diagram that illustrates one example method of manufacturing a chocolate delivery system for delivering a combination of dietary supplements and/or pharmaceuticals in accordance with the present invention.

### DETAILED DESCRIPTION

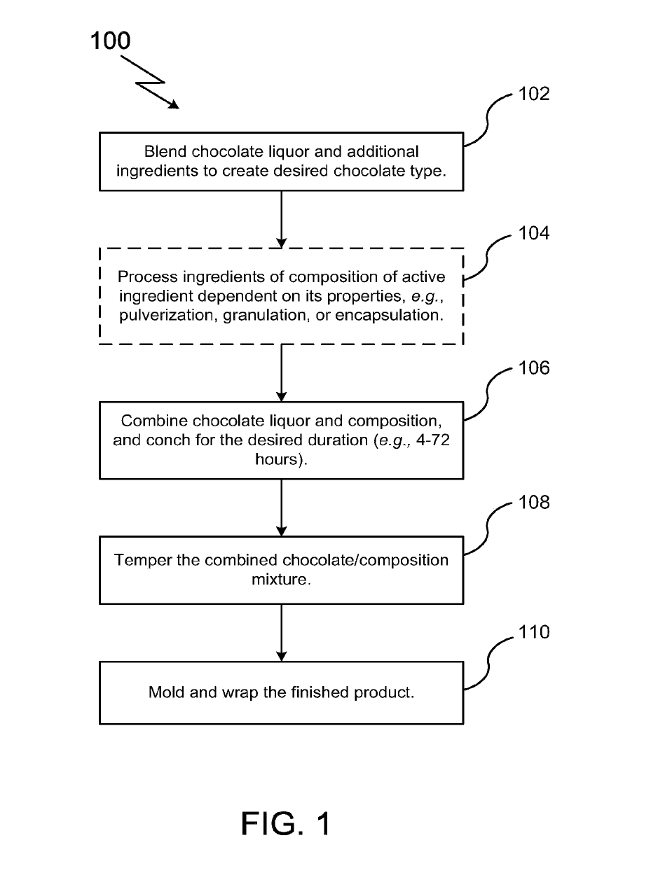

The present invention, as shown in FIG. 1, relates to a method of manufacturing a chocolate delivery system, in particular, a delivery system that is a chewable, solid chocolate candy designed to enhance delivery of the dietary supplements, pharmaceuticals, and/or other active ingredients contained therein. In general, the delivery system includes an active ingredient selected by the manufacturer to provide the desired effect, and chocolate as the delivery vehicle that contains the combination of dietary supplements for delivery. The active ingredient may include, for example, a combination of dietary supplements, that is, two or more ingredients selected from a group consisting of botanicals, vitamins, minerals, nutraceuticals, amino acids, soluble and insoluble fibers, prebiotics, probiotics, fatty acids, digestive enzymes, proprietary dietary compounds, or any other health promoting ingredient; or a pharmaceutical compound that includes an OTC or prescription drug.

FIG. 1 illustrates a method 100 of manufacturing a chocolate delivery system of the present invention. In general, the method of manufacturing the chocolate delivery system involves three main phases: (a) blending; (b) conching; and (c) tempering.

In the first phase of blending 102, the chocolate liquor made from the cacao nibs, which is a combination of cocoa butter (usually 50-60%) and cocoa solids, is blended with additional ingredients to obtain the desired individual chocolate recipe for the delivery vehicle. In general, dark chocolate consists of sugar, cocoa butter, cocoa liquor, and vanilla (optional). An emulsifying agent such as soy lecithin may be added to improve the texture of the chocolate.

In other implementations, the chocolate liquor may be sugar-free, made from natural and/or artificial sugar substitutes such as, for example, saccharin, aspartame, sucralose, neotame, acesulfame potassium, stevia, xylitol, sorbitol, maltitol, lactitol, or any other suitable sugar substitute.

In optional step 104, the appropriate dosage of the desired dietary supplements may undergo additional processing before being mixed with the blended chocolate and going to the conching phase 106. In the conching phase 106, the blended chocolate may be placed in a container filled with metal beads, which act as grinders, or in more modern conches, a rotary container with mixing blades. In general, in the conching phase, the blended chocolate mass is kept in a liquid state by frictional heat while it is “kneaded” at controlled temperatures (generally around 110-140° F.), which process may continue for four to six hours for lower-quality chocolate and seventy-two hours or longer for high-quality chocolate. The purpose of the conching is to reduce the size of the particles in the chocolate mass so as to produce a chocolate that is perfectly smooth and thus more desirable to the consumer. Generally, this requires reducing the size of the particles in the chocolate to approximately 18-20 microns in size. Additionally, the conching process is believed to improve the taste of the chocolate by removing harsh-tasting components and developing the more desirable flavor components of the chocolate. Thus, the duration of the conching process is dependent on many factors and varies with the type of chocolate desired to be produced and the quality of the original ingredients.

Therefore, dependent on the properties of the active ingredient to be added to the chocolate, it may be necessary to process one of the active ingredients if its particle size is too large for the conching process. For example, if the ingredient is in a solid powder form, it may be advantageous to pulverize it into a finer powder with a uniform particle size, while at the same removing any moisture in the ingredient. If the active ingredient is ground extremely fine, this may reduce the conching time. On the other hand, for other ingredients, it may be necessary in order to insure an even distribution of the ingredient throughout the chocolate mass, to granulate the ingredient, i.e., collect particles together by creating bonds between them, prior to its being conched with the chocolate liquor.

If the ingredient is in the form of an oil extract, such as, for example, chamomile extract, it would not be necessary for this ingredient to go through step 104, but such an ingredient may go directly to the conching process 106. Additionally, in the case of probiotics, prebiotics, and digestive enzymes, encapsulation may be required before the conching process.

In step 106, the appropriate dosage of the desired ingredients is added to the refined and blended chocolate from step 102, and conching of this mixture continues for the desired duration. As an alternative to conching (or as a supplement), an emulsification process may be utilized, in which case there may be less of a need to pre-process any of the ingredients.

In the next phase of tempering 108, the conched chocolate is heated, cooled, and then reheated to obtain the proper crystalline form and a uniform texture and appearance. In the final step 110, the tempered chocolate may be poured into molds of various sizes and shapes for candy bars or individually-wrapped pieces, which may then be wrapped and packaged for distribution to wholesale and retail outlets. The tempered chocolate may also be formed into large blocks, which may then be stored and/or transported to confectionary makers or other manufacturers who reheat and reprocess the chocolate to make their own products for their customers. These confectionary makers and other manufacturers may also purchase tempered chocolate that does not contain any active ingredient and produce their own chocolate delivery systems by repeating steps 104 through 110 of method 100.

One implementation discloses a chocolate delivery system comprising a chewable, solid dark chocolate candy and a plurality of dietary supplements that are uniformly dispersed throughout the chocolate candy. In general, the composition of dietary supplements comprises at least two ingredients selected from the group consisting of botanicals (which includes herbs), vitamins, nutraceuticals, i.e., extracts of food purported to have a medicinal effect on human health, amino acids, probiotics, prebiotics, fibers, and proprietary dietary supplements. In some instances, a composition of several ingredients may be considered a single ingredient. For example, a traditional well-known Chinese botanical may itself contain numerous ingredients and therefore can be considered a single ingredient. Another example is WellTrim™, a weight-loss nutraceutical formulated from a proprietary blend of standardized botanical extracts.

An example of an implementation is a composition formulated to assist the consumer with weight management that includes iron, calcium, vitamin B6 and B12, and WellTrim™. This composition may be implemented in individually-wrapped dark chocolate candies having 60% cacao mass, 8 grams in size, each candy containing the following formulation:

TABLE A
WEIGHT MANAGEMENT FORMULA

| Ingredients | Content (by Weight) |
|---|---|
| Intense Dark Chocolate | 91.39% |
| (60% cacao) | |
| Vitamin B6 | 0.03% |
| Vitamin B12 | 0.02% |
| Calcium | 0.04% |
| Cocoa Butter | 5.22% |
| Coffee flavor | 0.05% |
| Vanilla flavor | 0.10% |
| Cream flavor | 0.10% |
| WellTrim ™ | 3.05% |

A second example of an implementation is a composition formulated for stress relief where each candy contains the following formulation:

TABLE B
STRESS RELIEF FORMULA

| Ingredients | Content (by Weight) |
|---|---|
| Ghana Dark Chocolate | 90.02% |
| (70% cacao) | |
| Vitamin C | 0.59% |
| Valerian Root | 2.99% |
| Chamomile | 1.20% |
| Cocoa Butter | 5.00% |
| Natural Blueberry flavor | 0.20% |

Again, the delivery system may be individually-wrapped dark chocolate candies having 70% cacao mass, 8 grams in size, although dark chocolate having 85% or more cacao may also be used as well as dark chocolate with as little as 60% cacao mass.

Another example of an implementation is a composition formulated as a multi-vitamin for daily wellness where each candy contains the following formulation:

TABLE C
MULTI-VITAMIN FORMULA

| Ingredients | Content (by Weight) |
|---|---|
| Barry Dark Chocolate | 93.29% |
| (60% cacao) | |
| Multivitamin formulation | 2.91% |
| Cocoa Butter | 3.09% |
| Soy Lecithin | 0.01% |
| Natural Vanilla flavor | 0.70% |

The delivery system may be individually-wrapped dark chocolate candies having 60% cacao mass, 8 grams in size. In this implementation, the Multivitamin formulation may include, as an example, 500 IU of Vitamin A, 60 mg of Vitamin C (as Ascorbic Acid), 400 IU of Vitamin D (as Cholecalciferol), 30 IU of Vitamin E, 2.5 mg of Niacin, 1 mg of Vitamin B-6, and 3 mcg of Vitamin B-12. As used herein, the compound formulations may be expressed in terms of grams or milligrams, but may also be expressed in active units, or international unit (IU).

Yet another example of an implementation is a calcium-enrich composition where each candy contains the following formulation:

TABLE D
CALCUIM-ENRICHED FORMULA

| Ingredients | Content (by Weight) |
|---|---|
| Barry Dark Chocolate | 79.11% |
| (65% cacao) | |
| Cocoa Butter | 11.87% |
| Calcium (as Calcium carbonate) | 8.70% |
| Natural Raspberry flavor | 0.22% |
| Soy Lecithin | 0.10% |

The delivery system may be individually-wrapped dark chocolate candies having 65% cacao mass, 7 grams in size. In this implementation, the calcium-enriched formulation may also contain antioxidants, including, as an example, 200 IU of Vitamin D (as Ergocalciferol).

Another implementation discloses a chocolate delivery system comprising a chewable, solid dark chocolate candy and at least one active pharmaceutical ingredient (API) that is uniformly dispersed throughout the chocolate candy. In one implementation, the chocolate delivery system may be used to deliver OTCs or the active ingredients thereof to treat symptoms of common illnesses. Such OTC drugs may include Benadryl®, Sudafed®, Claritin®, Maalox®, Mylanta®, Tums®, Pepcid® AC, Monistat®, Ex-Lax®, Imodium® A.D., Robitussin®, Chloraseptic®, Thera-flu®, Alka-Seltzer, Motrin®, Dramamine®, and the like, in liquid or powder form.

For example, in one implementation of a chocolate delivery system for the treatment of heartburn, a single piece of chocolate candy having 65% cacao mass and a weight of, as an example, 7-10 grams, may contain 10 mg of famotide, 800 mg of calcium carbonate, and 165 mg of magnesium hydroxide, which is the equivalent of one gelcap of Pepcid® Supreme®. Thus, in a single piece of candy of 10 grams, the API would constitute approximately 10.0% by weight. However, the amount of the API added to the candy formulation must be balanced to insure that the eventual candy product will taste good as well as retain the desired texture. So, depending on the API being delivered, larger pieces of candy may be required or other flavorings and sweeteners may need to be added to the chocolate to mask the API in the more bitter formulations. For example, the serving size could be that of a standard size candy bar, e.g., 1.45-1.55 oz. (or 41-43 g) that contains the same amounts of the API.

In another implementation, the active ingredient may include a prescription drug. Such prescription drugs such may include Lipitor®, Singulair®, Lexapro, Plavix®, Morphine, Hydrocodone (Vicodin®), Demerol®, Codeine, Diazepam (Valium®), Penicillin, Prevacid®, Allegra-D®, Celebrex®, Crestor®, Cialis®, Valtrex®, Ambien CR®, Viagra®, Flomax®, Prozac®, and the like, in liquid or powder form.

As for the dosage, pharmaceutical compounds are generally expressed in terms of grams or milligrams, but may also be expressed in active units, or international unit (IU). As used herein, a “pharmaceutical compound” or “drug” shall include, but is not limited to, any drug, hormone, peptide, nucleotide, antibody, or other chemical or biological substances used in the treatment or prevention of disease or illness, or substances which affect the structure or function of the body.

In addition to these OTC and prescription drugs, the active pharmaceutical ingredient in the chocolate delivery system may also include nutraceuticals, i.e., extracts of food purported to have a medicinal effect on human health, such as botanical and herbal extracts and antioxidants. Also included are food supplements such as probiotics, prebiotics, vitamins and co-vitamins, minerals, amino acids, fibers, enzymes, or any other health promoting ingredient.

Thus, the chocolate delivery system may include one or more active ingredients that vary in the type and relative weight of the ingredients, with the only limitation being that the chocolate candy retains its flavor, appearance, texture, and smoothness, and that the active ingredient and any other supplemental ingredients be uniformly distributed throughout the delivery system. The delivery vehicle may further include natural colors, natural and artificial flavors, and preservatives. Additionally, the chocolate delivery system may take the form of a dark chocolate coating that is applied over another type of candy, such as caramel or nougat, i.e., a typical candy bar.

As used herein, the term “cacao mass,” also called “chocolate liquor,” refers to the liquid or paste produced when unroasted or roasted cocoa bean nibs are ground. This may include both cocoa solids (i.e., the non-fatty portion of the cocoa bean nibs) and additional cocoa butter added by the chocolate manufacturer to manipulate the finish and texture of the final product. Typical cocoa liquor contains about 50%-58% fat and about 42%-50% cocoa solids, by weight.

The foregoing description of implementations has been presented for purposes of illustration and description. It is not exhaustive and does not limit the claimed inventions to the precise form disclosed. Modifications and variations are possible in light of the above description or may be acquired from practicing the invention. The claims and their equivalents define the scope of the invention.

What is claimed is:

1. A chewable composition comprising:
a dark chocolate formed or molded solid candy; and
a plurality of dietary supplements selected from a group consisting of botanicals, vitamins, minerals, nutraceuticals, probiotics, prebiotics, fibers, fatty acids, amino acids, food supplements and any other health promoting ingredient, and any combination thereof.

2. The chewable composition of claim 1, wherein the plurality of dietary supplements includes at least one botanical and at least one vitamin.

3. The chewable composition of claim 2, wherein the at least one botanical is Valerian Root and chamomile extract and the at least one vitamin is Vitamin C.

4. The chewable composition of claim 1, wherein the plurality of dietary supplements includes at least one weight suppressant and at least one vitamin.

5. The chewable composition of claim 1, wherein the plurality of dietary supplements include at least 300 mg of Calcium.

6. The chewable composition of claim 1, wherein the solid candy contains at least 60% cacao mass.

7. The chewable composition of claim 1, wherein the solid candy contains at least 85% cacao mass.

8. The chewable composition of claim 1 further comprising at least one active pharmaceutical ingredient (API).

9. The chewable composition of claim 8, wherein the at least one API is an over-the-counter drug.

10. The chewable composition of claim 8, wherein the at least one API is a prescription drug.

11. The chewable composition of claim 8, wherein the at least one API further includes at least one composition selected from a group consisting of botanicals, vitamins, minerals, nutraceuticals, probiotics, prebiotics, fibers, fatty acids, amino acids, food supplements and any other health promoting ingredient, and any combination thereof.

12. The chewable composition of claim 1, wherein the solid candy contains chocolate liquor, cocoa butter, and natural cane sugar.

13. The chewable composition of claim 12, further including soy lecithin and vanilla.

14. A chewable composition comprising:
a dark chocolate formed or molded solid candy; and
at least one active pharmaceutical ingredient (API).

15. The chewable composition of claim 14, wherein the at least one API is an over-the-counter drug.

16. The chewable composition of claim 14, wherein the at least one API is a prescription drug.

17. The chewable composition of claim 14, wherein the at least one API further includes at least one composition selected from a group consisting of botanicals, vitamins, minerals, nutraceuticals, probiotics, prebiotics, fibers, fatty acids, amino acids, food supplements and any other health promoting ingredient, and any combination thereof.

18. The chewable composition of claim 14, wherein the solid candy contains chocolate liquor, cocoa butter, and natural cane sugar.

19. The chewable composition of claim 18, further including soy lecithin and vanilla.

20. The chewable composition of claim 14, wherein the solid candy contains at least 60% cacao mass.

21. The chewable composition of claim 14, wherein the solid candy contains at least 85% cacao mass.

22. A method of manufacturing a dark chocolate delivery system, the method including:
blending selected cacao nibs with additional ingredients to produce a chocolate liquor;
conching the chocolate liquor together with at least one active ingredient; and
tempering the conched chocolate.

23. The method of claim 22, further including the steps of:
forming the tempered chocolate into a molded shape; and
wrapping the molded candy.

24. The method of claim 23, wherein the additional ingredients further include natural cane sugar, soy lecithin, and vanilla.

25. The method of claim 24, wherein the at least one active ingredient includes a plurality of dietary supplements selected from a group consisting of botanicals, vitamins, minerals, nutraceuticals, probiotics, prebiotics, fibers, fatty acids, amino acids, food supplements and any other health promoting ingredient, and any combination thereof.

26. The method of claim 22, wherein the at least one active ingredient is an over-the-counter drug.

27. The method of claim 22, wherein the at least one active ingredient is a prescription drug.

28. The method of claim 22, wherein the at least on active ingredient includes any combination of dietary supplements and pharmaceuticals.

29. The method of claim 22, wherein the chocolate liquor is formulated to produce a chocolate having at least 60% cacao mass.

30. The method of claim 22, wherein the chocolate liquor is formulated to produce a chocolate having at least 85% cacao mass.

31. The method of claim 22, wherein the additional ingredients further include natural cane sugar, soy lecithin, and vanilla.

32. The method of claim 25, wherein the plurality of dietary supplements includes at least one botanical and at least one vitamin.

33. The method of claim 32, wherein the at least one botanical is Valerian Root and chamomile extract and the at least one vitamin is Vitamin C.

34. The method of claim 25, wherein the plurality of dietary supplements includes at least one weight suppressant and at least one vitamin.

35. The method of claim 25, wherein the plurality of dietary supplements include at least 300 mg of Calcium.

36. A method of manufacturing a dark chocolate delivery system, the method including:
reheating a block of tempered dark chocolate;
adding at least one active ingredient to the reheated chocolate;
blending the at least one active ingredient into the reheated chocolate; and
tempering the blended chocolate.

37. The method of claim 36, wherein the step of blending includes conching the reheated chocolate containing the at least one active ingredient.

38. The method of claim 36, wherein the step of blending includes using an emulsifying agent to blend the reheated chocolate containing the at least one active ingredient.

39. The method of claim 38, wherein the emulsifying agent is soy lecithin.

40. The method of claim 36, wherein the at least one active ingredient includes a plurality of dietary supplements selected from a group consisting of botanicals, vitamins, minerals, nutraceuticals, probiotics, prebiotics, fibers, fatty acids, amino acids, food supplements and any other health promoting ingredient, and any combination thereof.

41. The method of claim 36, wherein the at least one active ingredient is an over-the-counter drug.

42. The method of claim 36, wherein the at least one active ingredient is a prescription drug.

43. The method of claim 36, wherein the at least one active ingredient includes any combination of dietary supplements and pharmaceuticals.

44. The method of claim 36, wherein the reheated chocolate is formulated to produce a chocolate having at least 60% cacao mass.

45. The method of claim 36, wherein the reheated chocolate is formulated to produce a chocolate having at least 85% cacao mass.

46. The method of claim 36, wherein the reheated chocolate further includes natural cane sugar, soy lecithin, and vanilla.

47. The method of claim 40, wherein the plurality of dietary supplements includes at least one botanical and at least one vitamin.

48. The method of claim 47, wherein the at least one botanical is Valerian Root and chamomile extract and the at least one vitamin is Vitamin C.

49. The method of claim 40, wherein the plurality of dietary supplements includes at least one weight suppressant and at least one vitamin.

50. The method of claim 40, wherein the plurality of dietary supplements include at least 300 mg of Calcium.
